(19) United States
(12) Patent Application Publication
(10) Pub. No.: US 2020/0008970 A1
(43) Pub. Date: Jan. 9, 2020

(54) Protective Oral Sex Face Mask

(71) Applicant: LeRoy Ace Parker, Castaic, CA (US)

(72) Inventor: LeRoy Ace Parker, Castaic, CA (US)

(21) Appl. No.: 16/137,203

(22) Filed: Sep. 20, 2018

Related U.S. Application Data
(60) Provisional application No. 62/763,834, filed on Jul. 5, 2018.

(51) Int. Cl. A61F 6/04; A41D 13/11; A41D 31/00

(52) U.S. Cl. CPC A61F 6/04; A41D 13/1107; A61F 2006/042; A41D 31/00; A41D 13/1161

(57) ABSTRACT

A protective oral sex face mask is described herein that is comfortable to wear, pleasing for a wearer to use, and acts as a prophylactic against STDs and other infectious agents. The face mask includes a body portion bounded by a top boundary, a bottom boundary, a first side boundary, and an opposing side boundary. The body portion further includes an interior surface touchable by a wearer's tongue, and an exterior surface touchable on a recipient. The interior surface is flavored and/or includes a flavored substance to provide a desired taste to the wearer while performing oral sex.

### CROSS-REFERENCE TO RELATED APPLICATIONS

This patent application claims priority to provisional patent application Ser. No. 62/763,834 filed on Jul. 5, 2018; the contents of which are incorporated by reference.

### FEDERALLY SPONSORED RESEARCH OR DEVELOPMENT

Not Applicable

### BACKGROUND OF THE INVENTION

Many sexually transmitted diseases (STDs) can be transmitted via oral sex. To fight the spread of STDs, dental dams or other physical barriers are recommended while performing oral sex. However, these conventional barriers may be uncomfortable to wear and hinder the performer's performance. In addition, these barriers are typically made of a material having an unappealing taste, which might discourage someone from using a protective barrier at all.

Thus, there exists a need for a protective oral sex face mask that is comfortable to wear, pleasing for a wearer to use, and acts as a prophylactic against STDs and other infectious agents.

### FIELD OF THE INVENTION

The present invention generally relates to a protective oral sex face mask to protect the wearer from sexually transmittable diseases, and more particularly, to a protective oral sex face mask having a flavored inside to enhance the experiece of the wearer while performing oral sex.

### SUMMARY OF THE INVENTION

The general purpose of the protective oral sex face mask, described subsequently in greater detail, is to provide a protective oral sex face mask which has many novel features that result in a protective oral sex face mask which is not anticipated, rendered obvious, suggested, or even implied by prior art, either alone or in combination thereof.

A protective oral sex face mask is provided herein that is comfortable to wear, pleasing for a wearer to use, and acts as a prophylactic against STDs and other infectious agents. The face mask includes a thin and flexible body portion having dimensions to cover a wearer's mouth. The body portion is bounded by a top boundary, a bottom boundary, a first side boundary, and an opposing side boundary. The body portion further includes an interior surface and an exterior surface, where the interior surface is touchable by the wearer's tongue and the exterior surface is touchable on a recipient. The face mask further includes a face attachment connected to the body portion and configured to secure the face mask to the wearer's face. A flavored substance is disposed on the interior surface to provide a desired taste to the wearer while performing oral sex on the recipient.

The body portion of the face mask is designed to loosely fit about the wearer's mouth to provide ample room for tongue movement. The loose fitting is measurable by a wearer's ability to deform as much of the body portion that corresponds to a range-of-motion of the wearer's tongue from the wearer's mouth.

To provide comfort to the wearer while wearing the mask, the top boundary, the bottom boundary, the first side boundary, and opposing side boundary are lined with a material softer than a material of the body portion to provide comfort to the wearer. This soft material may include a textile such as cotton.

The body portion is further dimensioned such that the top boundary is wearable beneath the wearer's nose and along the wearer's top lip, the body portion drapes down across the wearer's lip area, and the bottom boundary runs under the wearer's chin. In other embodiments, the body portion is further dimensioned such that the top boundary is wearable over the wearer's nose, the body portion drapes down across the wearer's lip area, and the bottom boundary runs under the wearer's chin.

The face attachments may include a set of loops, a set of strings, or a strap. The face attachment may include an adjustment feature to tighten or loosen the mask on the wearer's face.

The flavored substance on the interior surface may have the flavor of at least one of a chocolate flavor, a strawberry flavor, or a vagina flavor. The exterior surface of the body portion may further include at least one of a stimulation substance, a lubricant, or a prophylactic substance.

Thus has been broadly outlined the more important features of the present protective oral sex face mask so that the detailed description thereof that follows may be better understood and in order that the present contribution to the art may be better appreciated.

Objects of the present protective oral sex face mask, along with various novel features that characterize the invention are particularly pointed out in the claims forming a part of this disclosure. For better understanding of the protective oral sex face mask, its operating advantages and specific objects attained by its uses, refer to the accompanying drawings and description.

### BRIEF DESCRIPTION OF THE DRAWINGS

In the figures, identical structures, element or parts that appear in more than one figure are generally labeled with a same numeral in all the figures in which they appear. Dimensions of components and features shown in the figure are generally chosen for convenience and clarity of presentation and are not necessarily shown to scale. The figures are listed below.

### FIGURES

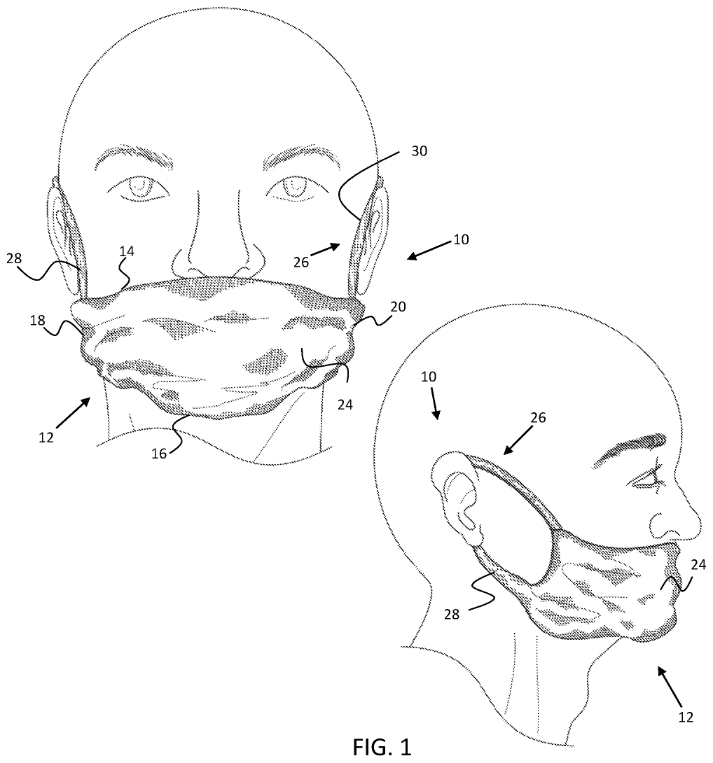

FIG. 1 depicts a front view and a side view of a protective oral sex face mask worn under a wearer's nose in accordance with embodiments of the invention.

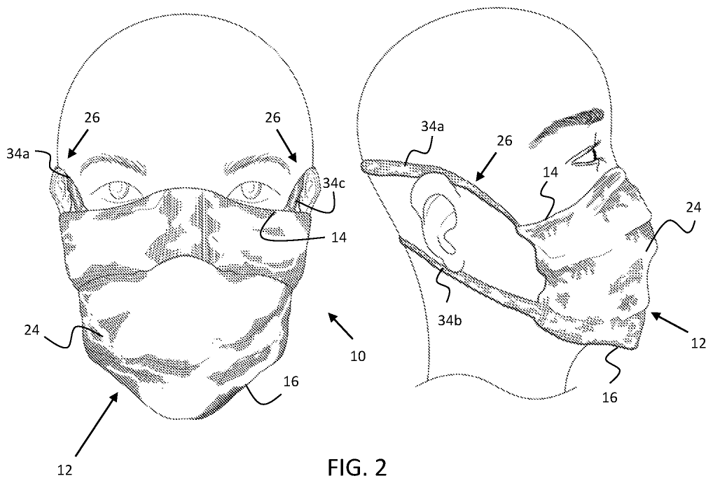

FIG. 2 depicts a front view and a side view of a protective oral sex face mask worn over a wearer's nose in accordance with embodiments of the invention.

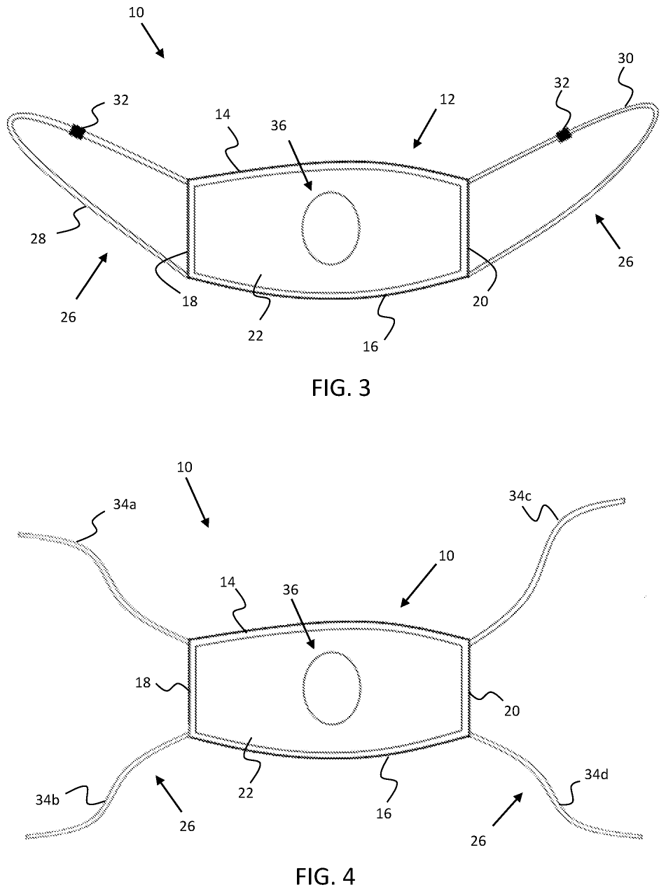

FIG. 3 depicts an interior view of a protective oral sex face mask having a first face attachment mechanism in accordance with embodiments of the invention.

FIG. 4 depicts an interior view of a protective oral sex face mask have a second face attachment mechanism in accordance with embodiments of the invention.

### DETAILED DESCRIPTION OF THE DRAWINGS

The present invention has utility as a protective oral sex face mask that is comfortable to wear, pleasing for a wearer to use, and acts as a prophylactic against STDs and other infectious agents. It should be appreciated that the protective face mask may be used by men and women alike. In preferred embodiments, the face mask is designed for oral sex on a vagina. The face mask is intended for use as a medical device to fight the transmission of sexually transmitted disease. Additional features of the mask promote the use of the medical device as further described below. The following description of various embodiments of the invention is not intended to limit the invention to those specific embodiments, but rather to enable any person skilled in the art to make and use this invention through exemplary aspects thereof.

With reference now to the drawings, and in particular FIGS. 1 through 4 thereof, examples of the instant protective oral sex face mask employing the principles and concepts of the present protective oral sex face mask and generally designated by the reference number 10 will be described.

Referring to FIGS. 1 through 4, a preferred embodiment of the present protective oral sex face mask 10 is illustrated, where FIG. 1 depicts a mask 10 worn below a wearer's nose and about the wearer's mouth, FIG. 2 depicts a mask 10 worn about a wearer's nose and mouth, FIG. 3 depicts an interior view of a mask 10 having loops to secure the mask 10 to the wearer's face, and FIG. 4 depicts an interior view of a mask 10 having strings to secure the mask 10 to the wearer's face. The protective oral sex face mask 10 generally includes a thin and flexible body portion 12 having dimensions to cover a wearer's mouth. The body portion 12 is bounded by a top boundary 14, a bottom boundary 16, a first side boundary 18, and an opposing side boundary 20. The body portion 12 further includes an interior surface 22 and an exterior surface 24, where the interior surface 22 is touchable by the wearer's tongue and the exterior surface 24 is touchable on a recipient. In one embodiment, the body portion 10 is dimensioned such that the top boundary 14 is wearable beneath the wearer's nose and along the wearer's top lip, where the body portion 12 drapes down across the wearer's lip area, and the bottom boundary 16 runs under the wearer's chin as shown in FIG. 1. In another embodiment, the body portion 12 is dimensioned such that the top boundary 14 is wearable over the wearer's nose, where the body portion 12 drapes down across the wearer's lip area, and the bottom boundary 16 runs under the wearer's chin as shown in FIG. 2. Having the body portion 12 worn over the wearer's nose may provide an additional level of protection by covering the wearer's nostrils.

The face mask 10 may be available in different sizes to accommodate differently sized wearer's, while in other embodiments the face mask 10 is dimensioned as a one-size-fits-all. The face mask 10 may also be available in two different models, an over-the-nose model and an under-the-nose model.

The face mask 10 further includes one or more face attachments 26 configured to secure the face mask 10 to the wearer's face. The face attachments 26 generally connect to the body portion 12 and attach with the wearer's head. In particular embodiments, with reference to FIGS. 1 and 3, the face attachments 26 includes a first loop 28 extending beyond the first side boundary 18 to loop around a wearer's first ear, and a second loop 30 extending beyond the opposing side boundary 20 to loop around a wearer's opposing ear. The loops (28, 30) may be made of an elastic material, such as elastic string. The loops (28, 30) may connect to the body portion 12 at either: the side boundaries (18, 20); the top boundary 14 and bottom boundary 16; and/or the corners formed between the top boundary 16, side boundaries (18, 20), and bottom boundary 16. In some embodiments, the loops (28, 30) are formed from a single elastic string that runs across the top boundary 14, around the wearer's first ear, across the bottom boundary 18, around the wearer's opposing ear, and back to the top boundary. It should be appreciated that there is enough string to ensure fitting around the wearer's ears and to make sure the mask 10 fits snuggly on the face. In a specific embodiment, at least one of the loops (28, 30) includes an adjustment feature 32 to adjust the fit of the face mask 10 on the wearer's face. For example, the adjustment feature 32 may be a clip that allows the wearer to adjust the overall length of the loop (28, 30) to tighten or loosen the loop (28, 30). Other examples of an adjustment feature 32 include a buckle, a clasp, or a pin and strap hole.

In another embodiment, with reference to FIGS. 2 and 4, the face attachments 26 includes a first string 34a and a second string 34b extending beyond the first side boundary 18, and a third string 34c and a fourth string 34d extending beyond the opposing side boundary 20. The face mask 10 may then be secured to the wearers face by one of the following. In one embodiment, the free ends of the first string 34a and the second string 34b are tied around a wearer's first ear, and the free ends of the third string 34c and the fourth string 34d are tied around the wearer's opposing ear. In another embodiment, the first string 34a and third string 34c run along opposing sides of the wearer's head and above the wearer's ears, the free ends thereof to be tied behind the wearer's head, and the second string 34b and fourth string 34d run along opposing sides of the wearers head and below the wearer's ears, the free ends thereof to be tied behind the wearer's head. The strings (34a, 34b, 34c, 34d) may be connected to the body portion 12 at either: the side boundaries (18, 20); the top boundary 14 and bottom boundary 16; and/or the corners formed between the top boundary 16, side boundaries (18, 20), and bottom boundary 16. In some embodiments, the first string 34a and third string 34c are the same string that runs along the top boundary 16 of the body portion 12, while the second string 34b and fourth string 34d are the same string that runs along the bottom boundary 18 of the body portion 12. The strings may further be made of an elastic material. In a particular embodiment, at least one of the strings (34a, 34b, 34c, and 34d) includes one of the aforementioned adjustment features 32.

It should be appreciated that other face attachments 26 may be used to secure the face mask 10 to the wearer's face. For example, the face attachments 26 may be one or more straps, like a belt, that straps about the wearer's head. The strap may be adjustable to tighten or loosen the mask 10 onto the wearer's face. Another example, includes a single elastic string that connects near a center region of the side boundaries (18, 20) and loops around the wearer's head.

In particular embodiments, the face mask 10 is designed such that the body portion 12 loosely fits about the wearer's mouth to provide ample room for tongue movement. The loose fitting of the body portion 12 may be measurable by a wearer's ability to deform as much of the body portion 12 that corresponds to a range-of-motion of the wearer's tongue from the wearer's mouth. In other words, the body portion 12 will loosely fit about the wearer's mouth such that the wearer's tongue can deform as much of the body portion 12 that the wearer's tongue can reach. This loose fitting may be achieved by an overall loose fitting of the mask 10 on the wearer's face, rather than a snug or tight fit. In other embodiments, the loose fitting may be achieved by a snug or tight fit on the wearer's face but the flexibility of the body portion 12 permits the loose fitting. In particular embodiments, the body portion 12 is made of a flexible and elastic material to permit ample room for tongue movement. This flexible and elastic material may be, for example, latex used for conventional condoms. In particular embodiments, the body portion 12 is extremely thin. In one embodiment, the body portion 12 is less than 10 mm, in another it is less than 5 mm, while in a further embodiment less than 1 mm. The thinness may contribute to the wearer's ability to move their tongue as well as increased sensation on the recipient.

To improve the comfort of the mask on the wearer's face, the top boundary 14, bottom boundary 16, and side boundaries (18, 20) are lined with a softer material than the remainder of the body portion 12. The soft material may include textiles having attributes known for their softness including cotton, silk, and cashmere. Other soft materials of polymeric origin may likewise be used. Preferably, the lining is approximately 1 inch wide along the boundaries (14, 16, 18, and 20).

In specific inventive embodiment, with reference to FIGS. 3 and 4, the interior surface 22 of the body portion 12 includes a flavored substance 26. The flavored substance 26 provides a desired taste to the wearer while the wearer performs oral sex on a recipient. The flavored substance 26 may be in solid or liquid form including a paste, gel, or powder. The flavored substance 26 may be a flavored material that makes up the body portion, such as flavored latex, while in other embodiments the flavored substance is a separate material attached to or incorporated with the interior surface 22. In some embodiments, the flavored substance 26 is initially covered by a seal to preserve the flavored substance 26, wherein the seal may be pealed or removed to expose the substance 26 when ready for use. Flavors for the flavored substance include strawberry, chocolate, vanilla, banana, or other flavors known in the art. In a particular embodiment, the flavored substance 26 is flavored like a vagina. If the flavored substance is separate from the material of the body portion 12 itself, then different combinations of flavors may be attached or incorporated on the interior surface 22. For example, one flavor may be accessible by the wearer in a first tongue position, and another flavor accessible by the wearer in a second tongue position. This may enhance the wearer's experience while performing oral sex. Overall, flavored substance 26 encourages the use of the face mask 10 and provides a degree of pleasure to the wearer of the mask 10 while in use.

The face mask 10 may further include a stimulation substance, lubricant, and/or prophylactic substance on the exterior surface 24 of the body portion 12. The stimulation substance or lubricant may provide sexual stimulation to the recipient during oral sex. A prophylactic substance may further reduce the probably of STD transmission. Examples of stimulation substances include synthetic or natural female/male arousal fluids, gels, or powders. The lubricant may be any safe sexual lubricant known in the art. The prophylactic substance may include antibiotics or other known infectious agent combatants. The stimulation substance, lubricant, and/or prophylactic substance may be initially covered by a seal that is subsequently removed for use as described above with regard to the flavored substance.

### OTHER EMBODIMENTS

While at least one exemplary embodiment has been presented in the foregoing detail description, it should be appreciated that a vast number of variations exist. It should also be appreciated that the exemplary embodiment or exemplary embodiments are only examples, and are not intended to limit the scope, applicability, or configuration of the described embodiments in any way. It should be understood that various changes may be made in the function and arrangement of elements without departing from the scope as set forth in the appended claims and the legal equivalents thereof.

What is claimed is:

1. A protective oral sex face mask comprising:
a thin and flexible body portion having dimensions to cover a wearer's mouth, said body portion bounded by a top boundary, a bottom boundary, a first side boundary, and an opposing side boundary, wherein said body portion further includes an interior surface and an exterior surface, the interior surface being touchable by the wearer's tongue and the exterior surface being touchable on a recipient;
a face attachment connected to the body portion and configured to secure the face mask to the wearer's face; and
a flavored substance on the interior surface to provide a desired taste to the wearer while performing oral sex on the recipient.

2. The face mask of claim 1 wherein the body portion loosely fits about the wearer's mouth to provide ample room for tongue movement, said loose fitting measurable by a wearer's ability to deform as much of the body portion that corresponds to a range-of-motion of the wearer's tongue from the wearer's mouth.

3. The face mask of claim 3 wherein the body portion at the top boundary, the bottom boundary, the first side boundary, and opposing side boundary is lined with a material softer than a material of the body portion to provide comfort to the wearer.

4. The face mask of claim 3 wherein the body portion is further dimensioned such that the top boundary is wearable beneath the wearer's nose and along the wearer's top lip, where the body portion drapes down across the wearer's lip area, and the bottom boundary runs under the wearer's chin.

5. The face mask of claim 4 wherein the face attachment comprises a first string and a second string extending beyond the first side boundary, and a third string and a fourth sting extending beyond the opposing side boundary, wherein the face mask is secured to the wearer's face by: a) an end of the first string tying with an end of the third sting in the back of the wearer's head, and an end of the second string tying with an end of the fourth string in the back of the wearer's head; or b) an end of the first string tying with an end of the second sting about a wearer's first ear and an end of the third string tying with an end of the fourth string about a wearer's opposing ear.

6. The face mask of claim 5 wherein the first string, second string, third string, and fourth string are elastic.

7. The face mask of claim 5 wherein the first string and third string are connected along the top boundary, and the second string and fourth string are connected along the bottom boundary.

8. The face mask of claim 4 wherein the face attachment portion comprises a first loop extending beyond the first side boundary to loop around a wearer's first ear, and a second loop extending beyond the opposing side boundary to loop around a wearer's opposing ear, wherein said loops secure the face mask to the wearer's face.

9. The face mask of claim 9 wherein at least one of the first loop and second loop are adjustable to loosen or tighten the face mask to the wearer's face.

10. The face mask of claim 4 wherein the face attachment comprises a strap to be strapped about the wearer's head, wherein said strap is adjustable to tighten or loosen the face mask to the wearer's face.

11. The face mask of claim 4 wherein the flavored substance has the flavor of at least one of a chocolate flavor, a strawberry flavor, or a vagina flavor.

12. The face mask of claim 4 wherein the body portion is made of an elastic polymeric material.

13. The face mask of claim 13 wherein the elastic polymeric material is latex.

14. The face mask of claim 4 further comprising at least one of a stimulation substance, a lubricant, or a prophylactic substance on the exterior surface of the body portion.

15. The face mask of claim 3 wherein the body portion is further dimensioned such that the top boundary is wearable over the wearer's nose, where the body portion drapes down across the wearer's lip area, and the bottom boundary runs under the wearer's chin.

16. The face mask of claim 15 wherein the face attachment portion comprises a first loop extending beyond the first side boundary to loop around a wearer's first ear, and a second loop extending beyond the opposing side boundary to loop around a wearer's opposing ear, wherein said loops secure the face mask to the wearer's face.

17. The face mask of claim 16 wherein at least one of the first loop and second loop are adjustable to loosen or tighten the face mask to the wearer's face.

18. The face mask of claim 15 wherein the flavored substance has the flavor of at least one of a chocolate flavor, a strawberry flavor, or a vagina flavor.

19. The face mask of claim 15 wherein the body portion is made of latex.
